(19) United States
(12) Patent Application Publication
(10) Pub. No.: US 2025/0299829 A1
(43) Pub. Date: Sep. 25, 2025

(54) SYSTEM AND METHOD FOR LONGITUDINAL ASSESSMENT OF A BIOMARKER, AND MRI APPARATUS

(71) Applicants: Siemens Healthineers AG, Forchheim (DE); Centre Hospitalier Universitaire Vaudois, Lausanne (CH)

(72) Inventors: Tobias Kober, Heidelberg (DE); Veronica Ravano, Ecublens (CH); Jonas Richiardi, Geneve (CH)

(21) Appl. No.: 19/085,043

(22) Filed: Mar. 20, 2025

(30) Foreign Application Priority Data
Mar. 20, 2024 (EP) 24164896.3

(51) Int. Cl. G16H 50/30; G06T 7/00; G16H 30/20

(52) U.S. Cl. CPC G16H 50/30; G06T 7/0016; G16H 30/20; G06T 2207/10088; G06T 2207/20076; G06T 2207/30096

(57) ABSTRACT

A system and a method for evaluating a longitudinal evolution of a quantitative biomarker measured for a biological object include receiving longitudinal measurements of the quantitative biomarker for the biological object. A statistical parameter, which characterizes a variability of the acquisition technique being used, is calculated for each measurement. The calculated statistical parameter is used for each obtained biomarker value for generating synthetic data having a distribution which follows the statistical distribution of possible values for the biomarker. The sampled values are fitted by using a fitting function for each bootstrapping fitting. A fitting parameter of the fitting function is extracted for each fitting. The longitudinal evolution of the quantitative biomarker is evaluated by statistically comparing the extracted fitting parameters against a reference value.

### CROSS-REFERENCE TO RELATED APPLICATION

This application claims the priority, under 35 U.S.C. § 119, of European Patent Application EP 24164896.3, filed Mar. 20, 2024; the prior application is herewith incorporated by reference in its entirety.

### FIELD AND BACKGROUND OF THE INVENTION

The present disclosure is directed, in general, to methods and systems for characterizing temporal changes of a quantitative biomarker measured for a biological object. More specifically, the present invention is directed to the monitoring of such a biomarker by using imaging techniques configured for imaging the biological object, for instance a brain tissue or lesion, the imaging technique being for instance Magnetic Resonance Imaging (MRI).

### DESCRIPTION OF THE RELATED ART

During radiological viewing, images (e.g., MRI images, x-rays, CTs, etc.) of one time point are often compared to those of another time point in order to track the evolution of a disease. For instance, the effectiveness of radiotherapy treatment is typically assessed by monitoring the tumor size over the duration of the therapy. Similarly, in multiple sclerosis (MS), the evolution of brain lesions is informative of disease activity. Specifically, the presence of “slowly expanding lesions” is a known characteristic of a specific disease progression in MS. Those lesions are characterized by a subtle volumetric expansion over time and their detection by the naked eye is typically challenging as it requires comparing lesion sizes in two different images displayed side by side where the differences might be small. Therefore, methods that provide an estimate of the longitudinal variation (i.e. the temporal variation) of relevant biomarkers over time (e.g., the size and shape of a tumor, of a multiple sclerosis lesion, a volume of a specific organ or brain region, but also for instance the average intensity/texture within a region of interest, or a non-imaging biomarker as blood-based biomarkers, etc.) are needed.

Various solutions have been proposed to improve the monitoring of biomarkers, notably by automating the comparison of biomarkers over time. However, the automatic estimation of a longitudinal evolution of biomarkers suffers from several technical challenges. In fact, the extraction of such biomarkers is sensitive to the variability of the underlying acquisition or measurement technique, as well as the processing techniques being used, which reduce the sensitivity to the true, physical change in the tissue of interest. The variability of automatic extraction methods can be caused by technical differences of the input data across different time points (e.g., in MRI, variability of the acquisition protocol and/or the employed hardware or different choice of energy level in CT, different echo angle in ultrasound etc.) which lead to different tissue contrast, signal-to-noise ratio and/or resolution. The resulting variability of the image biomarker extraction algorithms, such as, for example, a lesion segmentation algorithm, can be caused by technical and physical limitations of the acquisition. An example for such a variability-inducing effect is partial volumes, where the intensity of an image voxel is determined by multiple tissue types present inside this voxel (e.g., 70% of a voxel can be composed of lesioned tissue and 30% of healthy tissue); these “mixed voxel” intensities are difficult to classify (e.g., in a segmentation problem “lesion tissue” vs. “healthy tissue”) and are hence susceptible to cause also very minor variations between time points.

Therefore, ways to extract longitudinal biomarkers while accounting for the variability of the underlying acquisition and biomarker extraction methods are needed in order to provide a robust estimate of a longitudinal evolution of a biological object and a quantitation of the error engrained in the (longitudinal) measurement.

### SUMMARY OF THE INVENTION

It is accordingly an object of the invention to provide a system and a method for longitudinal assessment of a biomarker, and an MRI apparatus, which overcome the hereinafore-mentioned disadvantages of the heretofore-known systems, methods and apparatuses of this general type and which automatically assess a longitudinal evolution of a monitored quantitative biomarker.

With the foregoing and other objects in view there is provided, in accordance with the invention, a method for quantifying or evaluating the longitudinal evolution of a quantitative biomarker measured for a biological object, the method comprising:
receiving or acquiring, for the biological object, longitudinal measurements of the quantitative biomarker. For instance, N measurements M_1, . . . , M_N, performed according to a same or different acquisition techniques typically implemented by one or several acquisition devices known in the art, like an MRI or CT apparatus, are received or acquired. In particular, each measurement M_i is performed at a timepoint T_i according to an acquisition technique A_i, wherein if i≠j, then T_i≠T_j∀i,j∈{1, . . . ,N}, wherein for each measurement M_i, a value V_i is obtained or measured for the biomarker, wherein N is an integer strictly greater than 1, and i=1, . . . , N. Preferentially, the method comprises also performing the longitudinal measurements;
calculating, for each measurement M_i, at least one statistical parameter P_i (e.g. a variability or reliability coefficient or an error coefficient) which characterizes a variability of the used acquisition technique A_i, the statistical parameter P_i being a measure of a statistical distribution of possible values for the biomarker when carrying out the measurement M_i according to the acquisition technique A_i. The statistical distribution might be expressed as a confidence interval including the possible values that might be taken by the biomarker when carrying out the measurement M_i according to the technique A_i. The possible values represent potential variations of the measured value V_i that could have been obtained when performing the measurement M_i according to the technique A_i, the statistical distribution showing their likelihood to happen or to be observed. For instance, the statistical parameter might be a variability coefficient that provides an estimation of a variation of the value (e.g. a reliability of the measured value or an error on the measured value, wherein the error comes for instance from the devices used for measuring the biomarker and/or from the method or technique used for extracting the value of the biomarker from the measurement) of the biomarker when carrying out the measurement M_i according to the acquisition technique A_i;
for each obtained or measured biomarker value V_i, using the calculated statistical parameter P_i for generating synthetic measurement data the distribution of which-called hereafter “synthetic distribution” follows the statistical distribution of possible values for the biomarker (i.e. shows a same distribution as the statistical distribution), wherein each synthetic measurement data represents a simulated value for the biomarker and is assigned the timepoint T_i at which measurement M_i giving rise to the value V_i was performed. That means, creating a synthetic distribution of synthetic values the dispersion of which is defined by the statistical parameter obtained by the prior step. Typically, the synthetic data are configured for populating an interval of biomarker values including (e.g. centered around) the obtained or measured value V_i of the biomarker. Preferentially, the synthetic data distribution is statistically centered around the measured value V_i;
performing several bootstrapping rounds, wherein, for each bootstrapping, one or several values among the measured value V_i and the biomarker simulated values generated for the measured value V_i are randomly sampled from the synthetic distribution for each timepoint T_i;
for each bootstrapping, fitting the sampled values by using a fitting function;
for each fitting, extracting a fitting parameter of the fitting function;
evaluating the longitudinal evolution of the quantitative biomarker by statistically comparing the extracted fitting parameters against a reference value, which is notably chosen in function of a null hypothesis and might correspond for instance to a healthy condition or state for the biological object. For instance, the reference value refers to a value that would be obtained for the fitting parameters when considering a null hypothesis;
preferentially, automatically outputting a result of the evaluation. Preferentially, the result might be displayed on an appropriate display. The result is typically a representation, e.g. a graph, of the longitudinal evolution of the biomarker, preferentially including the reference value for enabling comparison. It is for instance the result of the statistical comparison with the reference value.

With the objects of the invention in view, there is also provided a system for quantifying or evaluating a longitudinal evolution of a quantitative biomarker measured for a biological object, the system comprising:
a first interface for receiving or acquiring, for the biological object, longitudinal measurements of the quantitative biomarker, wherein N measurements M_1, . . . , M_N, are received or acquired, wherein each measurement M_i is performed at a timepoint T_i according to an acquisition technique A_i, wherein if i≠j, then T_i≠T_j∀i,j∈{1, . . . ,N}, wherein for each measurement M_i, a value V_i is obtained (or measured) for the biomarker, wherein N is an integer strictly greater than 1, and i=1, . . . , N. Preferentially, ∀i,j∈{1, . . . ,N}, the acquisition technique A_i is the same as the acquisition technique A_j, i.e. the acquisition techniques A_1, . . . ,A_N are one and a same acquisition technique. Alternatively, it exists at least one acquisition technique A_j that is different from the acquisition technique A_i, with i≠j and i,j∈{1, . . . ,N}.;
a memory for storing the N measurements and the associated biomarker values; and
a control unit including a processor, the control unit being configured for carrying out the steps of the previously described method.

The system might be used for longitudinal monitoring of the quantitative biomarker. In particular, the system might be part for instance of an MRI or CT apparatus configured for performing the longitudinal measurements and automatically outputting the result of the evaluation according to the previously described method.

Dependent claims present further advantages of the invention.

### DESCRIPTION OF EMBODIMENTS

The present invention might be applied to any biomarker that is a measurable quantity typically representing a biological state or condition of a biological object, like a brain. The biomarker might be for instance:

a physical characteristic of the biological object or of a region of interest within the biological object, like a volume of the region of interest and/or its size and/or shape;
a measurable physiological characteristic of the region of interest or of the biological object;
a measurable biochemical characteristic of the region of interest or of the biological object;
or a combination of the latter. For instance, the biomarker might be a lesion volume. In such a case, the acquisition technique may include a segmentation of lesions appearing in the biological object. For this purpose, a segmentation algorithm might be used by the system according to the invention for identifying voxels, within the biological object, where a lesion is located. Known in the art techniques for segmenting a biological object might be used. The statistical parameter might be then a measure of the variability of results outputted by the segmentation algorithm. The statistical parameter can also be calculated by performing test-retest experiments for the acquisition technique A_i used for acquiring the measurement M_i. The estimated variability of each acquisition technique A_i encompasses notably the physical setup used for performing the measurement (e.g., as well as hardware or software resources) and the method, e.g., algorithm, used for extracting the value of the biomarker from a performed measurement. The statistical parameter defines the variability of obtained measured values when repeating the acquisition technique A_i for a same biological object. This enables analyzing changes more robustly that show in the biomarker values over time.

In particular, the present concept works for different acquisition techniques, like MRI acquisition techniques, X-ray acquisition techniques, computed tomography acquisition techniques, ultrasound acquisition techniques, or other acquisition techniques which enable to perform longitudinal measurements of a biomarker, and the variability of which might be estimated, using for instance test-retest reliability measurements, or other methods capable of providing an error estimate on extracted or measured values for the biomarker.

The fitting function enables modeling of the longitudinal changes of the quantitative biomarker while accounting for the reliability of the underlying biomarker acquisition technique, e.g. the extraction technique or method used for obtaining the biomarker value from each measurement. The fitting function might be a linear function, or any appropriate polynomial regression. In particular, statistically comparing the extracted fitting parameters against a reference value may include calculating a median estimate of the fitting parameters and testing the median estimate against a null hypothesis. This enables implementation of a statistical significance test providing insight on the statistical significance of the obtained result.

Preferentially, the fitting function is configured for fitting a first set of temporally consecutive timepoints independently from a second set of temporally consecutive timepoints, distinct from the first set, in order to independently evaluate, for the first set and the second set, a respective longitudinal sub-evolution of the quantitative biomarker. This provides the advantage of quantifying for instance the effect on the biomarker values of a chemical compound administered to, or a physical action performed on, the biological object that takes place between the first set and second set of timepoints. In particular, the reference value is either a value (e.g., if a null hypothesis is “no change”, wherein changes are quantified via the value of a slope extracted from the fitting function, then the reference value would be “0”, i.e. “slope=0”), or a distribution extracted from a set of measurements M′_1, . . . ,M′_K, called reference set, wherein each measurement M′_r, with r=1, . . . , K and K>1, has been performed at a timepoint T′_r. The distribution may come from the set of measurements M′_1, . . . , M′_K performed:
(i) for the biological object for which the measurements M_1, . . . , M_N were performed (called hereafter the tested biological object), wherein the set of measurements M′_1, . . . , M′_K are acquired at a different time period compared to the measurements M_1, . . . , M_N, that is the set of consecutive timepoints T′_1, . . . , T′_K, is different from the set of consecutive timepoints T_1, . . . , T_N. This can be used for showing an evolution of the biomarker with respect to a pre versus post treatment;
(ii) for another biological object or a group of other biological objects, e.g. healthy biological objects, wherein the set of consecutive timepoints T′_1, . . . , T′ K, might be the same or different from the set of consecutive timepoints T_1, . . . , T_N;
(iii) for the tested biological object, but using a different or several different acquisition techniques which serve as reference acquisition technique(s), and which are in the same time period as the time period including the timepoints T_1, . . . , T_N, the set of timepoints T_1, . . . , T_N being for instance identical to the set of timepoints T′_1, . . . , T′_K. This enables for instance to compare different acquisition techniques.

In other words, the set of consecutive timepoints T′_1, . . . , T′_K, might be different from the set of consecutive timepoints T_1, . . . , T_N, notably when they relate to measurements performed for the same biological object, but also if they relate to a different biological object, or might be the same as, or cover a same period of time as, the consecutive timepoints T_1, . . . , T_N, notably if they relate to measurements made for a different biological object, or if they relate to measurements made using a reference acquisition technique. The reference set serves as a set of reference for the measurement of the value of the biomarker. For instance, the reference set may include measurements performed for a healthy biological object whose longitudinal evolution of the biomarker has to be compared to the longitudinal evolution obtained for the biological object for which the measurement M_1, . . . , M_N were taken (e.g. if a slope is extracted from the fitting function, then the distribution might represent estimated slope values obtained for a control group of biological objects), or measurements performed for the biological object at a different period of time corresponding to the timepoints T′_1, . . . , T′_K with respect to T_1, . . . , T_N. Advantageously, this enables for instance to quantify the evolution of a biomarker before and after a treatment.

Other features which are considered as characteristic for the invention are set forth in the appended claims.

Although the invention is illustrated and described herein as embodied in a system and a method for longitudinal assessment of a biomarker, and an MRI apparatus, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a flowchart illustrating a method for evaluating a longitudinal evolution of a quantitative biomarker according to the invention;

FIG. 2 is a perspective view of a biological object and a block diagram of a system for evaluating a longitudinal evolution of a quantitative biomarker according to the invention;

FIG. 3 is a graph representing values of a quantitative biomarker as a function of time; and

FIG. 4 is a graph illustrating a result obtained via the method according to the invention.

### DETAILED DESCRIPTION OF THE INVENTION

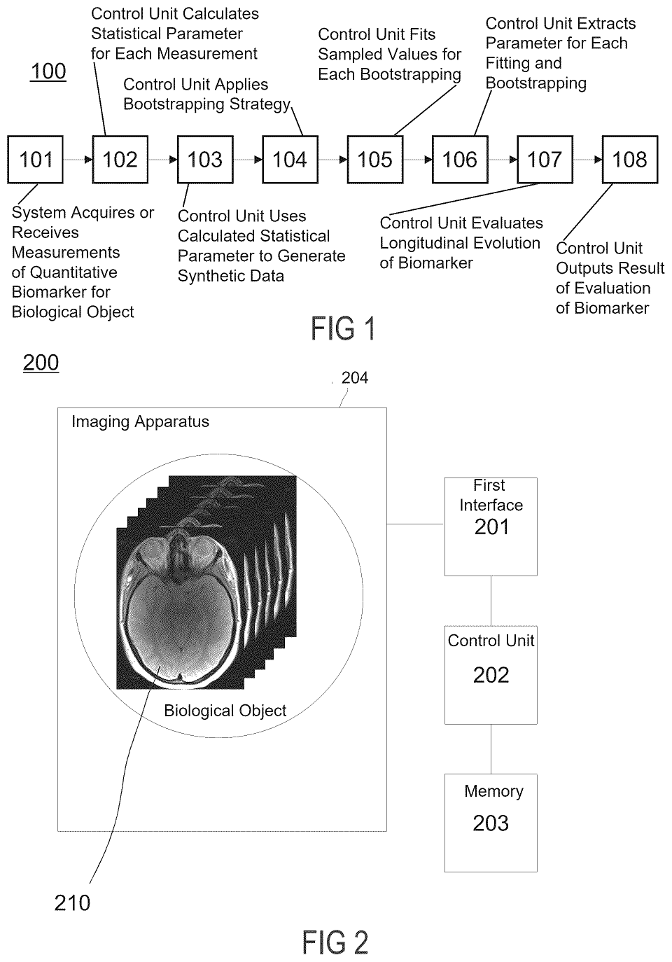

Referring now to the figures of the drawings in detail and first, particularly, to FIGS. 1 and 2 thereof, there is seen a preferred embodiment of the method according to the invention which will be described in more detail, wherein FIG. 1 illustrates the different steps of a method 100 carried out by a preferred embodiment of a system 200 according to the invention which is illustrated by FIG. 2.

The preferred embodiments illustrated in FIGS. 1 and 2 will be described in the context of lesion monitoring. However, the proposed invention can be applied to any quantitative biomarker. For example, the proposed method can be used to estimate differences in relaxometry measures over time (e.g., T1, T2 relaxation times), or any other biomarker (blood markers, cognitive tests, etc.), as long as an estimate of the reliability of cross-sectional measures (i.e. the statistical parameter) can be determined, e.g. via a test-retest setup as illustrated in the preferred embodiments, for generating then the synthetic data.

At step 101, the system 200 acquires or receives N measurements of the quantitative biomarker for a biological object 210. The N measurements are longitudinal measurements acquired or received via a first interface 201 of the system 200. The latter includes a control unit 202, connected to the first interface 201, and configured for processing the N measurements. Typically, the acquired or received measurements might be stored in a memory 203 of the system 200 according to the invention. In particular, the system 200 may include an imaging apparatus 204 for imaging the biological object 210, for instance a brain as illustrated in FIG. 2. Preferentially, the system 200 is an MRI apparatus configured for acquiring images of the biological object 210, wherein the images include voxels having an intensity which represents a measurement of the biomarker, and wherein the MRI apparatus is configured for automatically extracting, from the images, a value for the biomarker for the biological object, or for a region of the biological object. According to the present invention, each measurement M_i has been performed at a timepoint T_i that is different from the timepoint at which another measurement has been performed. Each measurement M_i might correspond for instance to an image acquired for the biological object at a timepoint T_i. The acquisition technique enables extraction, for each measurement M_i, of a value V_i for the quantitative biomarker, wherein the value V_i has been typically measured for a region of the biological object, or for the whole biological object.

At step 102, the control unit 202 calculates, for each measurement M_i, i.e. for each timepoint T_i, a statistical parameter, e.g. a variability coefficient that is an estimate of a variation of the measured value V_i, when extracting or measuring the value V_i via the acquisition technique. The statistical parameter is for instance a measurement uncertainty or error estimate that characterizes a statistical dispersion of the measured values V_i that would be obtained for instance when repeating the measurement for the same biological object in the same conditions several times. For example, if the biomarker is a lesion volume that is monitored by acquiring images of the biological object at different timepoints, e.g. by an MRI or CT apparatus, then the value of the volume might be obtained by segmenting the lesion in the acquired images using an existing segmentation algorithm, resulting, for each of the timepoints, to a respective lesion volume estimate. Instead of simply comparing the lesion volume estimates obtained for the different timepoints, the present invention proposes to use a measure of variability of the segmentation algorithm to model synthetic data in a synthetic distribution preferentially centered around the measured value of the lesion volume. This allows consideration of the reliability of the segmentation algorithm (which can vary based on differences in processing and imaging techniques) by providing a “certainty” margin around the originally measured lesion volume.

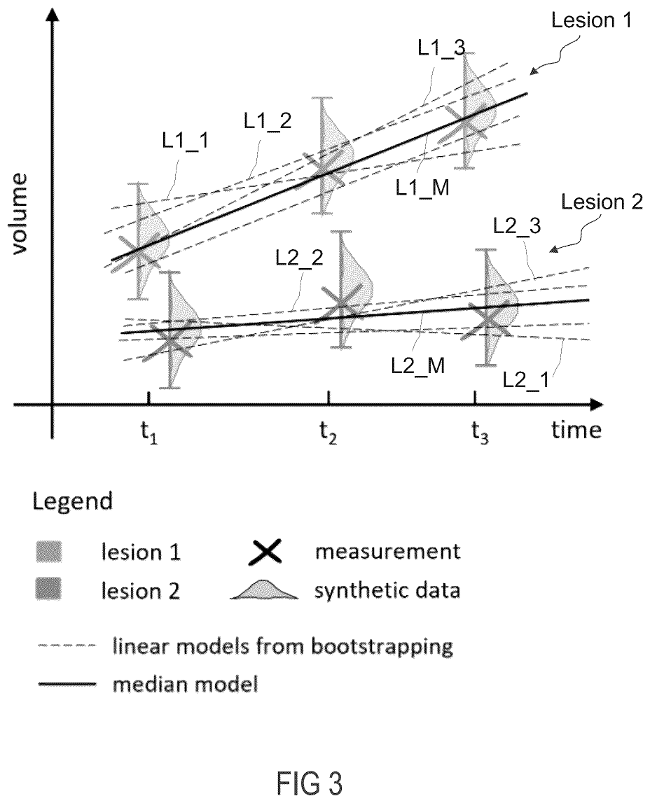

Indeed, at step 103, the control unit 202 uses the calculated statistical parameter for generating the synthetic data. The synthetic data are simulated or calculated data, while the measured value V_i is a real value that has been measured for the biological object. For instance, if the statistical parameter, i.e. the reliability, of the segmentation algorithm estimated in a test retest setting is ±5% and the measured lesion volume is 3 mL, then the control unit 202 might be configured for generating a normal distribution of K synthetic volume lesion values in the interval [3−(5%·3); 3+(5%·3)] mL, which is, in this case, naturally centered around the measured lesion volume, i.e. the 3 mL. This procedure is repeated for all measurements in all available time points. This is better illustrated in FIG. 3, wherein a first set of measurements is made for a first lesion (Lesion 1) and a second set of measurements is made for a second lesion (Lesion 2), wherein each measurement (represented by a cross x) is made at a timepoint t_i. The extracted biomarker is the volume of the lesion, the graph of FIG. 3 showing for each lesion, the evolution of the value of the biomarker (i.e. the lesion volume) as a function of time. For each measurement, synthetic data are generated around the measured lesion volume, wherein the values taken by the synthetic data follow a known-in-the-art distribution, e.g. the normal distribution, around the real value that has been measured, and which is illustrated by a “curve” around the measured value “x” in FIG. 3. In the example of FIG. 3, the synthetic data was generated as a normal distribution centered around the measured lesion volume, and with a width determined by the statistical parameter estimated in a test-retest experiment, giving rise to one hundred synthetic values generated for each measured lesion volume in each time point. While it is proposed herein to use a test-retest experiment to estimate the statistical parameter associated with the extraction of the imaging biomarker (lesion volumes), other measures of the statistical parameter, i.e. of the reliability of the biomarker measurements, might also be used. For instance, reliability measures might be estimated by comparing the output of the considered acquisition technique with a ground truth value. In the example of lesions, this could be achieved by measuring an estimation error in lesion volume when extracted using an automated segmentation algorithm using MRI data, with respect to a ground truth physical measure of ex-vivo histopathological samples. Alternatively, in the present context of lesion volume, lesion segmentations could be generated with built-in uncertainty estimates, using for instance Bayesian neural networks, which could then be used for calculating the statistical parameter.

At step 104, the control unit 202 is configured for applying a bootstrapping strategy, wherein, for each timepoint, one or several values among the measured biomarker value and the generated synthetic data for the measured biomarker value are randomly sampled. This means that for each timepoint t_i shown in FIG. 3 for the same biomarker (e.g. for the lesion volume of Lesion 1), one or several values among a set of values including the real and the synthetic data obtained for the timepoint are randomly selected. This corresponds to one bootstrapping, and such bootstrapping is performed several times (e.g. P times), resulting thus, for each biomarker, in several (e.g. P) sets of data, wherein each set of data includes, for each timepoint, one or several randomly selected set values for the biomarker.

At step 105, the control unit 202 is configured for fitting, for each bootstrapping, the sampled values. For example, for each of the several (e.g. P) sets of data, the randomly selected values of the biomarker as a function of time are fitted by a fitting function. As illustrated in FIG. 3, the fitting function might be a linear regression, wherein the different regression lines L1_1, L1_2, L1_3 and L2_1, L2_2 and L2_3 represent respectively for the first lesion and the second lesion different fittings obtained via a linear regression. Several regression lines might thus be obtained for each lesion of FIG. 3, e.g. 100 regression lines, using randomly sampled lesion volumes from the synthetic distributions in each timepoint.

At step 106, the control unit 202 extracts, for each fitting, and thus for each bootstrapping, at least one fitting parameter that will be used for evaluating longitudinal changes of the biomarker. The fitting parameter can be thus considered as a longitudinal change estimate for the measured biomarker. According to FIG. 3, the fitting parameter is for instance the slope of the different regression lines L1_1, L1_2, L1_3 obtained for the first lesion (Lesion 1), and L2_1, L2_2 and L2_3 obtained for the second lesion (Lesion 2) by fitting the sampled values with a linear regression. The slope of each regression line represents the lesion enlargement (or shrinkage) over time. Instead of a slope, other types of estimates for evaluating longitudinal changes can be defined, including for instance a computation of differences between sampled values, an estimation of a slope from fitting a polynomial line, or any other type of parameter estimated using function fitting (like fitting an exponential).

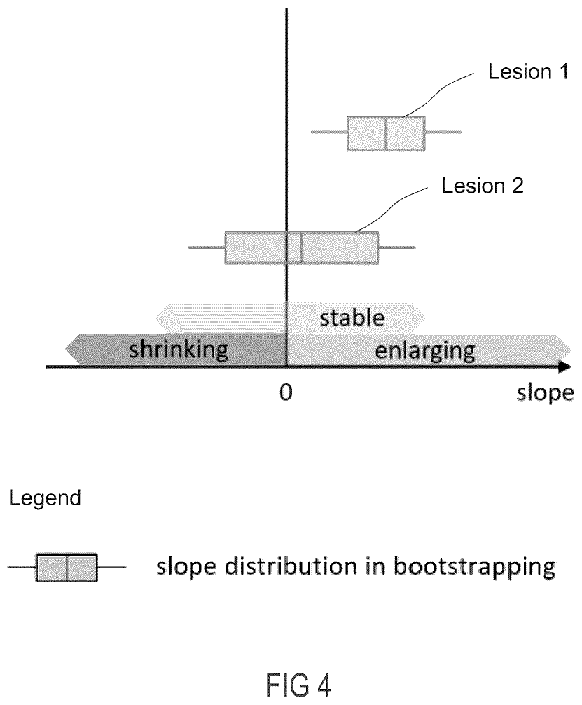

At step 107, the control unit 202 evaluates the longitudinal evolution of the biomarker by comparing the extracted fitting parameters against a reference value. In particular, the control unit 202 may calculate a median value of the at least one fitting parameter. This is illustrated in FIG. 3 by the lines L1_M and L2_M the respective slope of which is a median or average value of the slope values of the regression lines L1_1, L1_2, L1_3 and L2_1, L2_2, L2_3, respectively. L1_M represents thus a median model for the longitudinal evolution of the values of the biomarker as a function of time for the first lesion, and L2_M represents another median model for the longitudinal evolution of the values of the biomarker as a function of time for the second lesion. The distribution of the so-extracted fitting parameter(s), e.g. the slopes, is preferentially tested statistically for each lesion, notably against the reference value. For instance, if at least 95% of the slopes extracted from bootstrapping are positive, the lesion is enlarging. Similarly, shrinking and stable lesions (those not showing any significant longitudinal change) might be identified too. This is illustrated in FIG. 4, wherein the distribution of the extracted fitting parameters, i.e. the obtained slope distribution for each lesion, is compared to or tested against a reference value assuming no changes, i.e. a Null Hypothesis. This enables getting insight to the statistical significance of the median estimate, i.e. the median value of the slope, characterizing the evolution of the biomarker with time.

At step 108, the control unit 202 may automatically output a result of the evaluation of the longitudinal evolution of the biomarker. For instance, the result might be a graph, as illustrated in FIG. 4, that is automatically displayed, notably on a display of the system according to the invention, or outputted via a second interface of the system 200, which can be the same as the first interface.

The present invention allows a comparison of the values taken by a biomarker as a function of time, while taking into account the variability of the acquisition or extraction technique used for enabling the longitudinal comparison. The result is a more robust estimate of longitudinal changes of the value of a biomarker as it considers the certainty margin around each cross-sectional measure. Further, the final result is obtained directly for the concerned biological object, and is not based on a comparison with values obtained for a group of similar or identical biological objects. In addition, the present invention enables an association with each outputted result, i.e. with each evaluation of longitudinal changes of a biomarker, a statistical significance level which informs as to whether an observed change is worth noting or might be due to technical variabilities. This gives more certainty to the recipient of the respective analysis and might be a great tool for helping the recipient to make a decision.

To summarize, the present invention discloses a system and a method for evaluating a longitudinal evolution of a biomarker, i.e. for quantifying changes of the value of the biomarker over time. It proposes to generate a synthetic distribution of synthetic data to mimic the certainty associated with cross-sectional measurements of the biomarker, to perform bootstrapping in order to generate a pool of estimates of longitudinal biomarker value changes which can then be statistically compared against a reference, e.g. against a null hypothesis, to evaluate or quantify the evolution of the biomarker value with time. In particular, the statistical comparison of the distribution of bootstrapping samples against the null hypothesis may provide a p-value associated with each longitudinal change estimate, e.g. each measured slope, enabling thus to quantify the statistical significance of each longitudinal change estimate.

1. A method for evaluating a longitudinal evolution of a quantitative biomarker measured for a biological object, the method comprising:
receiving, for the biological object, longitudinal measurements of the quantitative biomarker, N measurements M_1, . . . ,M_N, being received, each measurement M_i, with i=1, . . . ,N, being performed at a timepoint T_i according to an acquisition technique A_i, when i≠j, then T_i≠T_j∀i,j∈{1, . . . ,N}, for each measurement M_i, and a value V_i being obtained for the biomarker;
calculating, for each measurement M_i, at least one statistical parameter characterizing a variability of the acquisition technique A_i being used, the statistical parameter being a measure of a statistical distribution of possible values for the biomarker when carrying out the measurement M_i according to the acquisition technique A_i;
for each obtained biomarker value V_i, using the calculated statistical parameter for generating synthetic data having a distribution following the statistical distribution of possible values for the biomarker, each synthetic data representing a simulated value for the biomarker and being assigned the timepoint T_i at which the measurement M_i was performed;
performing a plurality of bootstrapping rounds, for each bootstrapping, one or a plurality of values among the measured value V_i and the biomarker simulated values generated for the measured value V_i being randomly sampled for each timepoint T_i;
for each bootstrapping, fitting the sampled values by using a fitting function;
for each fitting, extracting a fitting parameter of the fitting function; and
evaluating the longitudinal evolution of the quantitative biomarker by statistically comparing the extracted fitting parameters against a reference value.

2. The method according to claim 1, which further comprises selecting the biomarker as:
a physical characteristic of the biological object or of a region of interest within the biological object; or
a measurable physiological characteristic of the region of interest or of the biological object; or
a measurable biochemical characteristic of the region of interest or of the biological object.

3. The method according to claim 1, which further comprises selecting a lesion volume as the biomarker, the acquisition technique A_i including segmenting lesions in the biological object by using a segmentation algorithm, and the statistical parameter being a measure of a variability of results outputted by the segmentation algorithm.

4. The method according to claim 1, which further comprises calculating the statistical parameter by performing test-retest experiments for the acquisition technique A_i.

5. The method according to claim 1, which further comprises carrying out the statistically comparing of the extracted fitting parameters against a reference value by calculating a median estimate of the fitting parameters obtained for the biological object and testing the median estimate against a null hypothesis.

6. The method according to claim 1, which further comprises configuring the fitting function for fitting a first set of consecutive timepoints T_i independently from a second set of consecutive timepoints T_i, distinct from the first set, to independently evaluate, for the first set and the second set, a respective longitudinal sub-evolution of the quantitative biomarker.

7. The method according to claim 1, which further comprises selecting the acquisition technique A_i as an MRI acquisition technique or an X-ray acquisition technique, or a computed tomography acquisition technique or an ultrasound-based acquisition technique.

8. The method according to claim 1, which further comprises selecting the fitting function as a linear regression.

9. The method according to claim 1, which further comprises:
(a) ∀i,j∈{1, . . . ,N}, providing the acquisition technique A_i to be the same as the acquisition technique A_j, or
(b) providing at least one acquisition technique A_j being different than the acquisition technique A_i, with i≠j and i,j∈{1, . . . ,N}.

10. The method according to claim 1, which further comprises providing the reference value as either a value, or a distribution extracted from a set of measurements M′_1, . . . , M′_K, called a reference set, each measurement M′_r with r=1, . . . , K, with K>1, having been performed at a timepoint T′_r, and the measurements M′_1, . . . ,M′_K having been performed for the biological object, or for another biological object.

11. A system for evaluating a longitudinal evolution of a quantitative biomarker measured for a biological object, the system comprising:
a first interface for receiving or acquiring longitudinal measurements of the quantitative biomarker for the biological object, N measurements M_1, . . . , M_N, being received or acquired, each measurement M_i being performed at a timepoint T_i according to an acquisition technique A_i, when i≠j, then T_i≠T_j∀i,j∈{1, . . . ,N}, and for each measurement M_i, a value V_i being obtained for the biomarker;
a memory for storing the N measurements M_i and their respective associated biomarker value V_i; and
a control unit including a processor, the control unit being configured for carrying out the method according to claim 1.

12. A Magnetic Resonance Imaging apparatus, comprising the system according to claim 11 configured for performing the N measurements.
